(19) United States
(12) Patent Application Publication
(10) Pub. No.: US 2008/0233577 A1
(43) Pub. Date: Sep. 25, 2008

(54) METHOD FOR PRODUCING LIGANDS, LIGANDS AND TEST KIT

(75) Inventors: JURGEN OBERSTRASS, Habichtswald (DE); Detlev Zwingmann, Marburg (DE); Bernhard Meisegeier, Veitshochheim (DE)

Correspondence Address: FLEIT GIBBONS GUTMAN BONGINI & BIANCO P.L., ONE BOCA COMMERCE CENTER, 551 NORTHWEST 77TH STREET, SUITE 111, BOCA RATON, FL, 33487, US

(73) Assignee: ANALYTICON BIOTECHNOLOGIES AG, Lichtenfels (DE)

(21) Appl. No.: 11/937,100

(22) Filed: Nov. 8, 2007

Related U.S. Application Data
(63) Continuation of application No. PCT/EP2006/003927, filed on Apr. 27, 2006.

(30) Foreign Application Priority Data
May 9, 2005 (DE) 10 2005 022 057.6

(51) Int. Cl. C12Q 1/68; G01N 33/566; G01N 33/00

(52) U.S. Cl. 435/6; 436/501; 436/94

(57) ABSTRACT

The invention relates to a method for producing ligands, in particular aptamers. With this method, a target substance is offered to a set of candidate ligands, and the unbonded ligands are separated out by a cross-flow filtration process. The retentate, which contains ligand-target substance complexes, then undergoes further continuous cross-flow filtration while chemical and/or physical parameters are being varied. After each variation of a parameter, those candidate ligands whose bond with the target substance was dissolved by the parameter variation are collected in separate fractions and separately multiplied.

### CROSS-REFERENCE TO RELATED APPLICATIONS

This application is based upon and claims priority from prior International Application No. PCT/EP2006/003927 filed on 27 Apr. 2006, which claims priority from German Patent Application 10 2005 022 057.6 filed on 9 May, 2005, the entire disclosures of which are hereby incorporated herein by reference.

### BACKGROUND OF THE INVENTION

1. Field of the Invention

The present invention generally relates to the field of ligands, and more particularly relates to ligands, producing ligands that are capable of binding with a target substance, and a diagnostic test kit comprising such ligands.

2. Description of Related Art

The invention relates to a method comprising the following steps for producing ligands that are capable of binding with a target substance:
Bringing a candidate set, consisting of a plurality of different candidate ligands, into contact with the target substance in order to permit binding to occur between the candidate ligands and the target substance,
Separating the candidate ligands that do not bind with the target substance from the candidate ligands that have bonded with the target substance, using a physical separation method, and discarding the separated candidate ligands,
Breaking the bond between the non-discarded candidate ligands and the target substance and
Multiplying at least one of the non-discarded candidate ligands.

The invention also relates to correspondingly produced ligands as well as a diagnostic test kit comprising such ligands.

A method of the type mentioned above is disclosed in EP 0533838 B1, a German translation of which has been published as DE 69128350 T2. The referenced document is concerned with the production of so-called aptamers. An aptamer is a specifically binding oligonucleotide and there is no fundamental restriction regarding the target substance that is specifically bonded by the oligonucleotide. It may be a sequence of nucleic acids, a protein, a cell surface element, or similar.

The production of oligonucleotides with a predetermined sequence of nucleic acids has long been known. However, it is difficult or even impossible to predict the binding properties, in particular specific binding properties, of a synthesized oligonucleotide. It is therefore customary, as described in EP 0533838 B1, to select desired aptamers from a large set of candidate oligonucleotides. For this purpose, the target substance of interest is offered to the candidate set under conditions that make it fundamentally possible for binding to take place. Next, the mixture of candidate ligands and target substance undergoes a physical separation process, for example gel electrophoresis. This method allows unbonded ligands to be separated from the ligand-target substance complexes. The unbonded oligonucleotides may be discarded because they demonstrably are unable to bind with the target substance and are thus eliminated as specific ligands. In a subsequent step, the bond of the ligand-target substance complexes is dissolved and the ligands are purified and multiplied. The latter is achieved in the case of oligonucleotides by employing customary and known multiplication methods. In order to improve the specificity of the aptamers that are found, the process described is repeated many times in the known methods, and in each case the previously multiplied oligonucleotides are used as the candidate set.

The known method, which is usually referred to as SELEX, has the disadvantage that even after multiple iteration of the method, the ligands that are finally found always consist of a set of different nucleotides. In order to obtain “pure” aptamer species it is therefore necessary to clone and sequence the members of the set in order to isolate them and then, on the basis of statistical considerations, to test one or more of the clones obtained to determine whether they have the desired binding capability and, if they do, to continue to use them. It is true that when this method is used it can be assumed, with a high degree of certainty, that the ligand found will bind with a high degree of affinity to the target substance, but it is nevertheless necessary to test the specificity of the ligand in subsequent cross reaction tests. If it turns out in such tests that the ligand found possesses undesirably low selectivity, the entire procedure must be repeated, without the operator having the opportunity to exert any specific influence on the selection process described above in order to arrive at a better result, i.e. a truly specific ligand.

DE 100 48 944 A1 discloses a method for producing aptamers in which the candidate set first undergoes electrophoresis. A fraction defined by a run-time parameter of the electrophoresis is isolated for further use. This fraction is then reacted with target molecules. When electrophoresis is repeated under the same conditions, the fraction defined by the same run-time parameter can be discarded because it was obviously not influenced by the reaction with the target substance, i.e. it failed in particular to bind with the target substance. Some or all of the other fractions which, on the other hand, show that they were influenced by the target substance, are grouped together to form a new candidate set that is again put through the above mentioned process. This method exhibits two major disadvantages. On the one hand, it takes a very long time to perform because of the very time-consuming electrophoresis that is repeated many times. On the other hand, right away in the first stage, i.e. when a fraction is selected from the candidate set, an arbitrary step occurs in which most of the candidate set is discarded for reasons that have nothing to do with the candidates' suitability to bind specifically with the target substance.

DE 100 49 074 A1 discloses a further method for producing aptamers in which the target substance is immobilized in a column. A candidate set is then made to run over the column so that the candidate ligands can bind with the immobilized target molecules and can themselves be immobilized. The path length of the candidate ligands in the column is interpreted as an indication of their corresponding affinity. Next, the column is isolated and individual ligand fractions are arranged according to their binding affinity. This method, too, has the disadvantage that it is extremely time-consuming and costly. Furthermore, the method works only for large amounts of candidate ligands, because the assumed correlation between affinity and path length is a product of the mass action law and thus, as a statistical statement, applies only in the case of large numbers of molecules. But precisely this hampers the formation of pure aptamer fractions.

U.S. Pat. No. 5,683,916 discloses a method for affinity purification of candidate ligands in a continuous cross-flow filtration process, wherein molecules of the target substance are immobilized in a filter membrane made up of hollow fibres, and the said molecules hold binding-capable ligands in the membrane so that they can be obtained by subsequent washing and elution. One disadvantage of this known method is that the filter membrane must be tailor-made for each specific application by carrying out special pre-treatment, in particular by immobilizing the target substance. This is an expensive and time-consuming process. Another disadvantage of the method is that no differentiation is made between candidate ligands that have the common characteristic of being able to bind with the target substance, but otherwise might have a different structure.

U.S. Pat. No. 5,872,015 also discloses a method for affinity purification of candidate ligands. Binding-capable candidate ligands are accumulated in a special device without immobilizing target substances. The accumulated candidate ligands are present as complexes bonded to the target substance. In order to differentiate between candidate ligands having high and low affinity to the target substance, elution is carried out in two steps using different buffer solutions. For further differentiation it is proposed that mass spectroscopic analysis be performed. It is a disadvantage of this method that the mass-spectroscopic analysis is very time-consuming and costly. In addition, such an analysis provides information only about the composition of the set of selected candidate ligands, but it does not allow the purity or homogeneity of the set to be improved.

U.S. Pat. No. 5,891,742 discloses a method for determining relative affinities of two components to a target substance. In this method, a component set containing both components is mixed with the target substance in two different proportions, and in each case the non-binding components are separated out from the two mixtures. Next, the two mixtures are examined by mass spectroscopic means and the relative affinities of the components are determined by comparing the mass spectra. This method is not suitable for affinity purification, in particular of a heterogenous candidate set.

U.S. Pat. No. 6,180,348 B1 discloses a method for identifying an aptamer. In this method, oligonucleotides of different sequences are in each case fixed on solid supports and jointly fixed to a base support. Next, a target substance that is immobilized on magnetic beads is made to react with the fixed oligonucleotides, and magnetic force is applied to separate from the base support those oligonucleotides whose interaction with the target substance is stronger than the interaction of their support with the base support. The need to have available sufficiently large quantities of pure oligonucleotides before performing this identification step is a serious disadvantage of this method. The oligonucleotides can be obtained by explicit synthesis, but this makes the selection process lengthy and inefficient.

Therefore, a need exists to overcome the problems with the prior art as discussed above.

### SUMMARY OF THE INVENTION

It is the task of the present invention to make available a method for producing ligands that overcomes the disadvantages of the state of the art methods and that is in particular suitable to arrive at specific ligands in a non-iterative fashion.

This task is solved, in conjunction with the features described in the preamble to claim 1, by using a continuous cross-flow filtration process to separate bonded and unbonded candidate ligands, wherein a filtration liquid containing the candidate ligands and the target substance is separated by means of a filter unit, over which the filtration liquid flows, into a retentate containing the target substance and the candidate ligands bonded thereto, on the one hand, and a filtrate containing the unbonded candidate ligands, on the other hand, and the filtrate is separated from the liquid stream, and the method is further characterized by the fact that after the unbonded candidate ligands have been discarded, and before the multiplication step is carried out, the following steps are performed:
Modification of at least one of the chemical and/or physical parameters that influence the binding between candidate ligands and target substance,
Separation, after each of several modification steps, of those candidate ligands whose bond with the target substance was dissolved as a result of the parameter modification, into a ligand fraction assigned to the respective modification step, and
Separate multiplication of at least one of the ligand fractions obtained.

It should be noted that the method of the invention can in principle be applied to any type of ligand. For example, it can be arranged that instead of or in addition to oligonucleotides, the candidate set can comprise nucleic acid-protein complexes, arrested translation complexes, phages, bacteria and/or eukaryotic cells. However, to simplify the description, the invention is explained below using oligonucleotides as the example. On the basis of these explanations, a person skilled in the art can easily apply the knowledge to other types of ligands.

### BRIEF DESCRIPTION OF THE DRAWINGS

FIG. 1 illustrates a schematic view of a device for carrying out the method according to the invention.

### DETAILED DESCRIPTION

According to the invention, provision is made for the candidate ligands and the target substance to undergo jointly a cross-flow filtration process, also known as overflow filtration. In this process a portion of the filtration liquid, referred to as the filtrate, is separated out by means of a filter unit, e.g. a filter membrane or a bundle of hollow fibres or similar, from the portion of the filtration liquid referred to as the retentate, which continuously circulates over the filter unit. Flow velocity, filtration volume, etc., can be adjusted in the manner with which a person skilled in the art is familiar, and according to the requirements of the specific application, by adjusting the equipment parameters such as pressure, pump speed, pore size of the filter unit, etc. In the case of the present invention, the adjustments are made in such a way that unbonded candidate ligands are separated out with the filtrate, whereas ligand-target substance complexes formed by a candidate ligand binding to a particle of target substance remain in the retentate. The unbonded candidate ligands separated in this way with the filtrate can be discarded because they are obviously not capable of binding with the target substance and thus cannot be considered as the desired ligands.

As explained, along with the target substance, all the candidate ligands that have become bonded to the target substance remain in the retentate. According to the invention, a further step is now provided in which at least one chemical and/or physical parameter that influences the binding between the candidate ligands and the target substance is modified. The modification can take place slowly and continuously or—preferably—in steps, and the step height and the direction of the change should be dimensioned in such a way that some of the ligand-target substance complexes are broken up so that free ligands and free particles of target substance are formed. Within the framework of the ongoing cross-flow filtration process, the freed candidate ligands enter into the filtrate which, however, unlike in the first process step described above, is not discarded but is taken up in a ligand fraction assigned to the current parameter modification step. It should be noted that the concept of the modification step or the step height in the case of a continuous parameter modification designates a predetermined parameter range, and all candidate ligands that have dissolved their bond with the target substance while passing through this range are taken up in a corresponding common fraction.

This fractionation step, i.e. the modification of a parameter that influences the binding, and the subsequent collecting of the detached ligands in a corresponding fraction, is repeated several times in order to obtain a plurality of ligand fractions having different binding properties with regard to the target substance. This is clearly evident in the various breaking conditions of the bond represented in the various fractions. It can therefore be assumed with great probability that the ligands having different binding properties, which are collected in various fractions, also differ structurally, i.e. for example in their sequence in the case of oligonucleotides. If the parameter modification is graduated sufficiently finely, it is thus possible, in contrast to the state of the art, to obtain almost pure fractions of candidate ligands that are all in principle capable of binding with the target substance. Depending on the planned use, the parameter to be modified may be a temperature, a pH value or a salt content of the filtration liquid. Concentrations of detergents, chaotropic agents, denaturing substances (e.g. formamide or urea) or competitive ligands may be varied as parameters.

In a final step, these fractions, or at least some of them, may be separately multiplied and their binding characteristics may be examined more closely in cross reaction tests or other tests in order to determine their specificity to the target substance. If the result of such a test is that the specifically examined ligand fraction does not meet the conditions required for a specific application, it is not necessary, as in the state of the art, to repeat the entire process; instead, it is sufficient to subject another fraction, previously obtained and multiplied, to the same test. In this way it can be ensured to the greatest possible extent that all the candidate ligands contained in the original set undergo an appropriate test for suitability. This is not the case in the current state of the art because there the process is subjected to substantially random selection mechanisms that can lead to a situation where, even after multiple reiteration of the entire process, certain candidate ligands never remain “winners” of the random selection process and therefore are never considered for concrete application tests. The method of the invention furthermore substantially accelerates the production of the desired ligands because, in one single pass through the process, a plurality of basically suitable ligand fractions are formed.

It is a further advantage of the method of the invention that, for each individual ligand fraction, precisely those conditions under which the bond with the target substance was dissolved are known. This additional physico-chemical information can be used by a person skilled in the art to give special consideration to certain ligand fractions for specific applications, or to exclude them. This, too, can be used for the more targeted development of specific active substances.

In a preferred embodiment of the invention, within the framework of the cross-flow filtration process, a volume of liquid corresponding to the separated filtrate and free of target substance and candidate ligands is supplied continuously or gradually to the filtration liquid. This means, in other words, that the separated volume of filtrate in the cross-flow circulation is replaced. As a result, on the one hand, the process of the invention can run almost indefinitely, thus permitting almost unlimited fine graduation of the resulting fractions. On the other hand, the volume replacement also leads to heavy dilution of the filtration liquid because the added liquid volume does not contain any candidate ligands or target substance. This dilution significantly improves the statistics with regard to pure fractions. In a typical application of the method of the invention to produce aptamers the candidate set contains approx. 10¹¹ different oligonucleotides. Of these, however, only about 10³, for example, bind with the target substance. The others should already be discarded in the first process step as being fundamentally unsuitable. It is clear that this goal is attained only in the rarest of cases, if only because of the unfavourable statistics. Instead, it must be expected that some of the non-binding candidate ligands remain in the retentate. If, however, the retentate is diluted in the following steps by the replenishing the volume as referred to above, ideally to the extent that the individual fractions contain individual ligand molecules, this statistical disadvantage can be overcome so that eventually pure or almost pure fractions are in fact obtained.

Within the framework of the multiplication step of the individual fractions, a preferred embodiment of the invention provides for the multiplied ligands to be given a marker. This may, for example, be a particle marker, say in the form of gold or latex pellets, a fluorescent marker, an enzymatic marker, or similar. In this way, the multiplied ligands of the individual fractions can be easily identified in a binding test that [is carried out] for example on a micro-array or a multiwell plate using immobilized target substance and/or competing binding substances.

In the concrete application of the method of the invention for producing aptamers the candidate set, as already explained, comprises oligonucleotides. For this purpose, preferably nucleotides that have one or more variable regions consisting of randomly modified nucleotide sequences bounded by constant regions on the 5′ and 3′ sides are used.

In this case, the variable regions comprise preferably at least 12-30 nucleotides. The constant regions comprise preferably 12-22 nucleotides. It is preferable that each of the variable regions should have a continuous structure. However, it has also proved advantageous to use variable regions that are interrupted once or several times by constant regions that in turn each comprise 2-5 nucleotides. These blocks of defined sequences ensure that preferred aptamers with specific secondary structures occur in the candidate mixture. For example, the formation of helical regions is preferred if a series of cytosins is introduced, or the selection of hairpin structures is preferred if the constant block contains a “tetraloop sequence”.

For the practical implementation of the cross-flow process when producing aptamers that bind, for example, to proteins or cells, it is advantageous to use a filter unit whose pore size retains the usually significantly larger proteins or cells, while the significantly smaller oligonucleotides, in unbonded form, can pass through the filter element and into the filtrate. In other cases, however, in which the ligands and the target substance particles are of comparable size, it has proved advantageous to bind the target substance to substrate particles which are large compared to the pore size of the filter unit. These may be, for example, gold, sepharose, agarose, latex or glass pellets that cannot pass through the filter unit. It should, however, be pointed out that this does not immobilize the target substance on the filter unit. Instead, the substrate particles, with the target substance particles bonded to them, flow freely in the retentate.

From the preceding explanation it is clear that ligands produced according to the invention may, on the one hand, be significantly cheaper than those produced according to the state of the art, and in addition may be produced in a more targeted manner with regard to specific applications, so that their optimization for a specific application can far exceed what has hitherto been achievable.

Correspondingly, it can be expected that diagnostic test kits that contain the ligands produced according to the invention will be cheaper and perform better than those used in the past.

However, it should be pointed out that the possible applications of the ligands of the invention are not limited to diagnostic in-vitro tests. Indeed, the ligands may be used in any type of binding-dependent detection process, e.g. also in the food or environmental sectors, or in phytopathology. Therapeutic applications and diagnostic procedures in living organisms are also conceivable.

Further advantages of the present invention are apparent from the following specific description and from the drawing, which shows:

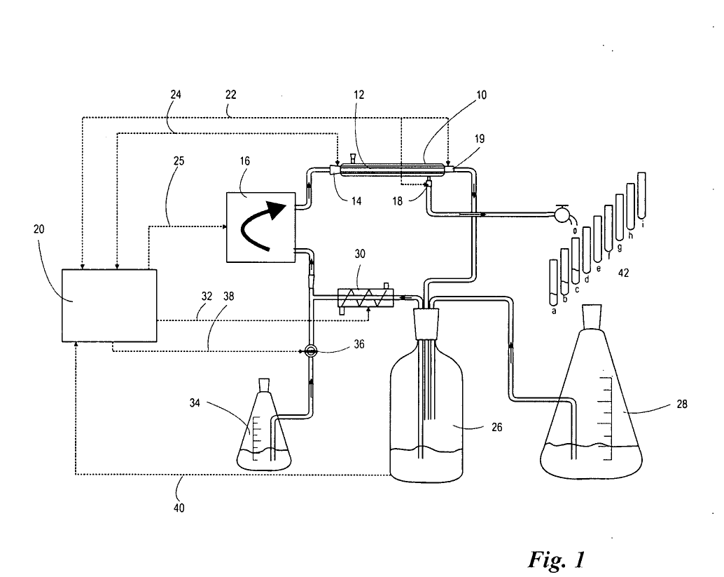

FIG. 1: a schematic view of a device for carrying out the method according to the invention.

FIG. 1 shows a schematic view of an apparatus for carrying out the method according to the invention, with reference to which the course of the procedure will be explained. The core of the apparatus is a cross-flow filtration unit 10, which is equipped in the usual manner with a filter membrane or a filter cartridge 12. The cross-flow device has an inlet 14 via which filtration liquid is supplied by means of a pump. The cross-flow unit 10 has two outlets 18 and 19. The filtrate outlet 18 serves to drain away that portion of the filtration liquid that has passed through the filter cartridge 12. On the other hand, the outlet 19 for the retentate drains away that portion of the filtration liquid that could not pass through the filter cartridge 12. The relative volumes of filtrate and retentate depend on the various setting parameters of the cross-flow unit 10. These include the performance of the pump 16, the characteristics of the filter cartridge 12 and the pressure ratios at the inlets and outlets 14, 18, 19. In order to permit the appropriate adjustment or regulation of the parameters, a control device 20 is provided that is connected to the pump 16 via measuring and control lines and also to pressure adjustment devices (not shown) in the cross-flow unit 10. The measuring and control lines are shown as dashed arrows in FIG. 1, with the heads of the arrows indicating the predominant direction of the information flow. For example, the measuring and control line 22 exchanges measuring and control data with pressure regulating devices at outlets 18 and 19, the measuring and control line 24 exchanges measuring and control data with a pressure adjusting device at inlet 14, and the control line 25 sends control signals to the pump 16.

The inlet to the pump 16 is connected with a filtration liquid reservoir 26 into which the retentate from outlet 19 also drains.

At the start of the process, the reservoir 26 is filled with filtration liquid to which are added the candidate set as well as the target substance. In this first process step a first binding reaction takes place between the target substance and those candidate ligands which are fundamentally capable of binding with the target substance. The filtration liquid thus contains ligand-target substance complexes, unbonded ligands and, as a function of the starting quantities used, also unbonded target substance.

This mixture is circulated through the cross-flow device 10 by means of the pump 16. The filter cartridge 12 is designed in such a way that unbonded ligands are able to pass through the filter and reach the filtrate outlet 18, while ligand-target substance complexes and possibly unbonded target substance remain in the retentate and are returned to the filtration liquid reservoir 26 via the retentate outlet 19. In this initial filtration step the filtrate can be fully discarded because the candidate ligands contained therein are obviously not capable of binding to the target substance.

In the particularly advantageous apparatus shown in FIG. 1, a further liquid reservoir 28 is provided in which filtration liquid without any candidate ligands or target substance is held in readiness. This liquid from the reservoir 28 is used to replace the volume lost by diverting the filtrate. In addition, the circulating mixture becomes correspondingly “diluted” and this has a positive effect on the statistics of the process.

Once the unbonded candidate ligands have been substantially removed from the mixture, a parameter that influences the binding between the candidate ligands and the target substance is varied in a subsequent step. This parameter may be, for example, the temperature of the circulating liquid. This is schematically shown in FIG. 1 by means of a temperature control coil 30, and the temperature is regulated by the control unit 20 via the measuring and control line 32. Alternatively, or in addition, the parameter to be varied can relate to the chemical conditions of the circulating liquid. The parameter could be for example the pH value, the urea concentration or specific salt concentrations. In FIG. 1 this is represented by the chemical reservoir 34 from which a titration solution is added to the circulating mixture via the valve 36. The valve is controlled by the control unit 20 via the control line 38. In FIG. 1 a general measuring line 40 is depicted between reservoir 26 and the control unit 20; this is intended to schematically represent the measurement of all the relevant parameters as well as the corresponding sensors. It goes without saying that a person skilled in the art will mount the appropriate sensors at the appropriate positions on the device and will perhaps combine several measuring lines. FIG. 1 is therefore to be understood as merely symbolic for representational purposes and not as a concrete plan for the design of a corresponding device.

By varying a parameter as described above, the binding conditions of the ligand-target substance complexes are modified, with the result that the bonds of some of the complexes are dissolved. The ligands that are released can then pass through the filter cartridge 12 and end up in the filtrate, which is no longer discarded in this step of the process but is collected in individual fractions that are given the reference numbers 42 a-i in FIG. 1. Next, the same or another parameter is further varied and the resulting filtrate is collected in a new fraction. This step can in principle be repeated as often as necessary, depending on how many parameter variation steps are required. The filtrate yielded in each variation step is collected in its own fraction so that, in effect, this yields a plurality of fractions 42 a-i, each of which can be assigned to a parameter variation step. As already explained, it is also possible to continuously vary the parameters, and this means that a parameter range can be assigned to each fraction.

Next, at least some of the 42 a-i fractions obtained can be multiplied and subjected to further tests to determine their suitability for specific, desired applications. As already explained in the general description, the multiplication may also comprise a marking of the ligands obtained in the fractions, and the suitability tests may comprise binding and cross reaction tests.

Of course, the exemplary embodiment explained in the drawing and the specific description is only an exemplary and illustrative embodiment of the present invention. In particular the design of the apparatus can be varied by a person skilled in the art in a wide variety of ways within the scope of the present invention. This holds true in particular for the adaptation to specific ligands that are preferably, but not necessarily, oligonucleotides, so that the oligonucleotide ligands which are finally obtained may be referred to as aptamers.

What is claimed is:

1. A method for producing ligands capable of binding with a target substance, comprising the following steps:
bringing a candidate set consisting of a plurality of different candidate ligands into contact with the target substance in order to permit binding between the candidate ligands and the target substance,
separating the candidate ligands that do not bind to the target substance, from the candidate ligands that are bonded to the target substance by using a physical separation process, and discarding the separated candidate ligands, wherein said physical separation process is a continuous cross-flow filtration process in which a filtration liquid containing the candidate ligands and the target substance is separated, by means of a filter unit over which the filtration liquid flows, into a retentate that contains the target substance and the candidate ligands bonded thereto, on the one hand, and into a filtrate containing the unbonded candidate ligands, on the other hand, the filtrate being separated out of the liquid flow,
breaking the bond between the non-discarded candidate ligands and the target substance by modifying at least one chemical and/or physical parameter that influences the binding between candidate ligands and target substance,
separating, after each of several modification steps, those candidate ligands whose bond with the target substance was dissolved by the parameter modification, into a ligand fraction associated with the respective modification step and
separately multiplying at least some of the ligand fractions obtained.

2. A method according to claim 1,
wherein the parameter modification takes place in steps.

3. A method according to claim 1,
wherein the parameter modified is the temperature, the pH value, the content of denaturing substances, a salt content or the presence of a competitive binding agent in the liquid.

4. A method according to claim 1,
wherein an amount of liquid free of target substance and candidate ligands that is equivalent to the separated-out filtrate is supplied continuously or gradually to the filtration liquid.

5. A method according to claim 1,
wherein the candidate ligands are combined with a marker during the multiplication step.

6. A method according to claim 1,
wherein the candidate set comprises oligonucleotides of various sequences.

7. A method according to claim 6,
wherein the candidate set comprises oligonucleotides that have one or more variable regions consisting of randomly modified nucleotide sequences which are bounded by constant regions on the 5′ and the 3′ sides.

8. A method according to claim 1,
wherein the candidate set comprises oligonucleotides of various sequences comprising oligonucleotides that have one or more variable regions consisting of randomly modified nucleotide sequences which are bounded by constant regions on the 5′ and the 3′ sides, wherein the variable regions comprise at least 12 to 30 nucleotides.

9. A method according to claim 1,
wherein the candidate set comprises oligonucleotides of various sequences comprising oligonucleotides that have one or more variable regions consisting of randomly modified nucleotide sequences which are bounded by constant regions on the 5′ and the 3′ sides, wherein each of the constant regions comprises 12 to 22 nucleotides.

10. A method according to claim 1, wherein the candidate set comprises oligonucleotides of various sequences comprising oligonucleotides that have one or more variable regions consisting of randomly modified nucleotide sequences which are bounded by constant regions on the 5′ and the 3′ sides, each of the variable regions having a continuous structure.

11. A method according to claim 1,
wherein the candidate set comprises oligonucleotides of various sequences comprising oligonucleotides that have one or more variable regions consisting of randomly modified nucleotide sequences which are bounded by constant regions on the 5′ and the 3′ sides, the variable regions being interrupted once or several times by constant regions that each comprise 2 to 5 nucleotides.

12. A method according to claim 1,
wherein the candidate set comprises nucleic acid protein complexes, arrested translation complexes, phages, bacteria or eukaryotic cells.

13. A method according to claim 1,
wherein the target substance is bonded to substrate particles that are large compared with the pore size of the filter unit.
